(19) United States
(12) Patent Application Publication
(10) Pub. No.: US 2020/0327558 A1
(43) Pub. Date: Oct. 15, 2020

(54) METHOD OF PREVENTING PHARMACEUTICAL FRAUD USING INTEGRATED IDENTITY MANAGEMENT

(71) Applicant: Spinal Guides Labs, LLC, CAROLINA, PR (US)

(72) Inventors: Albert Davydov, Carolina, PR (US); Peter Usov, Belle Mead, NJ (US)

(21) Appl. No.: 16/890,477

(22) Filed: Jun. 2, 2020

Related U.S. Application Data
(63) Continuation-in-part of application No. 16/879,176, filed on May 20, 2020.
Application No. 16/879,176 is a continuation-in-part of application No. 16/383,698, filed on Apr. 15, 2019.

(51) Int. Cl. G06Q 30/00; G16H 10/60; G16H 20/10; G16H 40/20; G06Q 50/26; G06F 16/25

(52) U.S. Cl. CPC G06Q 30/0185; G16H 10/60; G06F 16/258; G16H 40/20; G06Q 50/265; G16H 20/10

(57) ABSTRACT

The method of preventing pharmaceutical fraud using a system of integrated unique identity management by generating a unique person identifier and using this unique person identifier to correspond with a governing entity for authorization to issue a prescription for a controlled substance. The authorization is only issued if no conflicts are found. The unique person identifier serves as a special patient tag with universal abilities allowing for the person's identity to be managed across various data sources, thus preventing the person from obtaining multiple or unauthorized prescriptions.

### RELATED APPLICATIONS

The present application is a continuation-in-part claiming priority to U.S. patent application Ser. No. 16/879,176 for SYSTEM AND METHOD OF INTEGRATED UNIQUE IDENTITY MANAGEMENT, filed on May 20, 2020 (pending), which was a continuation-in-part claiming priority to U.S. patent application Ser. No. 16/383,698 for SYSTEM AND METHOD OF INTEGRATED PATIENT UNIQUE IDENTITY MANAGEMENT, filed on Apr. 15, 2019 (pending).

### BACKGROUND

The present invention is in the field of systems and methods for identifying personal data. More specifically, the present invention is a system and method of using the unique identification to prevent unauthorized distribution of controlled substances through legal channels through pharmacies.

The following background information may present examples of specific aspects of the prior art (e.g., without limitation, approaches, facts, or common wisdom) that, while expected to be helpful to further educate the reader as to additional aspects of the prior art, it is not to be construed as limiting the present invention, or any embodiments thereof, to anything stated or implied therein, or inferred thereupon.

Personal data in many industries, and electronic health care data in healthcare, in particular, are always tried to be linked together across multiple systems, including electronic health records (EHRs), patient registries, claims databases. In general, each system assigns its own identifier to each person whose data it maintains. This process makes it difficult to track data across multiple systems and identify duplicate data entry when different systems are linked. Efforts to address this challenge are complicated by the need to protect data privacy and security.

Personal Data Global Unique Identification System (PDGUIS) is defined as the system with abilities to ascertain a distinct, unique identity as expressed by an identifier that is unique within the scope of the exchange network, given characteristics about that identity such as name, date of birth and gender. PDGUIS utilizes the process to accurately and appropriately identify, track, manage, link individuals and their digitized health or other information, often within and across multiple electronic systems.

A related idea is the concept of personal identity integrity defined as the accuracy and completeness of data attached to, or associated with an individual. Efficient identity management leads to high identity integrity. One of the solutions is to assign a personal identifier tag (PIT), to each person's unique body part patterns (some human body part patterns can be subject for unique identification) and share it among data storage facilities. There are biometric data collection devices utilized based on this attribute for security identification and authentication purposes. However, each individual device system contains certain percentage of inaccuracy and has capability issues.

For example, fingerprint method, the oldest and the most popular biometric technology is only 99.3 percent reliable. Fingerprint databases of U.S. government agencies alone store more than 200 million records. However, quality issues respective to degrading prints, or development of skin callouses over time during hard and repetitive labor complicate the read. More effective, in terms of accuracy were the palm vein biometric devices. However, they are also problematic due to high pattern template size and high cost. Other methods and devices may seriously limit the possibilities of their applications in global and nationwide identity systems.

In addition, a simple generation of global unique identification numbers requires a centralized generation system and a distribution system for these numbers. There are also problems of assigning the unique numbers to individuals. There are also problems arising from assigning different numbers to the same individual, or for different individuals' assignment the same number. In such cases, these numbers cannot be used as incidental because they are not synchronized. For example, medical institutions erroneously assign duplicate numbers to one person. Or, in a personal identity fraud personal identification can be stolen and an identifier (ID) can be assigned to someone else.

Using above methods simultaneously create the person's (or in healthcare, patient's) global unique identification system. It combines biometric data to verify a person's (patient's) identity and assigns the unique individual number to make the system more accurate and flexible. This approach eliminates most problems described above because the biometrics can prevent individuals from registering in the global unique identification system multiple times and, vice versa, an individual unique number of a person can be retrieved from the global system using biometric data.

Unfortunately, this method also has serious limitations for globalization. Biometric patterns, or templates, must be stored on a central server during enrollment. Direct matching process of biometric patterns or templates may take a long time. A personal biometric template captured by the biometric device must be sent to the central server that requires good network, internet connectivity and sufficient hardware operating resources.

Accuracy in this process depends on several other factors such as false acceptance rate (FAR), false reject rate (FRR), error rate, identification rate, etc. Significant disadvantage of those limitations is the fact that the system cannot operate with institutions that have no internet connection or internet connection is unstable. In this case, it would be advantageous to have a system that can generate a unique person's IDs from personal identification locally without any connection to the central database. Such a system will be more reliable and easier to use in the PDGUIS.

The unique ID can be formed based on individual data of a person such as first, middle and last names, birthday in combination with personal unique body part pattern, or template. On the other hand, personal unique body part pattern, or template can be supplied with an individual person's, or as in healthcare, Patient Individual Tag (PIT), based on individual data of a person.

This approach reduces the personal IDs data fetch duration from the national database and increase accuracy in storing data. However, limitations for wide application of this system can be lack of accuracy of the processing and measurements issues and a high cost of instruments.

The present invention of the method and system offers a simple, cost-effective and portable solution with high accuracy, fast search speed for biometric patterns, or templates in the global database storage.

Today, common biometric approaches include facial recognition, fingerprinting and iris scanning. These systems are limited due to their complexity, infringement on privacy, cost, or portability issues.

The present invention describes using ear biometrics and it is not limited to it. Any biometrics can be used as well. Ear biometrics has proven to be a unique and viable solution. It does not require particular actions, such as scanning of a part of a body over an authentication device which makes it easier to conduct continuous authentication. The system works everywhere, even when the person is moving. Ears are remarkably consistent unlike faces, they do not change shape with different expressions, or age and remain fixed in the middle of the side of the head against a predictable background.

In the present invention, we have developed an identification algorithm that also shows good scalability of recognition rate with size of a dataset. We then conducted re-recognition and then identification and statistical analyses to identify the accuracy and replicability of our method. In conclusion, the bend, or flexure of the ear helix was found to be the most reliable anatomical structure that could be served as the base for re-identification.

It is known that single ear identification rate might vary from 90% to 99.5% for image ray transform methods. In the present invention, our approach is to use both, left and right ears together and our rate of identification was up to 100%.

The present invention is a simple, cost-effective and portable identity identification method in the global system that allows managing identity across countrywide data sources. It minimizes data management expenses such as storage requirements for the device itself. The personal data will be stored locally. There is only a simple web camera and a simple image processing application, or a lightweight image processing application necessary.

The present invention can be used in many industries. The examples could be healthcare global ID verification for medical equipment, electronic healthcare (EHR) patient record management and management of patient identity across data sources. The national patient identification system would identify patients, link patient medical records, and allow broad sharing, monitoring, research and analysis of public using computerized medical records linked through the Nationwide Health Information Network (NHIN). Other examples may include government organizations, national identity applications, financial institutions and other facilities requiring high security storage. Personal data information exchange enables information sharing across disparate health care applications. It also can be used as a platform for development of biometric identification application for desktop and mobile devices. System can meet the minimum hardware requirements for current mobile devices on the market today and can be used on many mobile devices such as Android, or the Apple iOS system, or a like.

One specific application of the inventive system and method is prevention of pharmaceutical fraud. Specifically, one way to obtain controlled substances from pharmacies is by using fake IDs. As an example, the NY Health Department's I-STOP/PMP—Internet System for Tracking Over-Prescribing—Prescription Monitoring Program Prescription Monitoring Program Registry, does not prevent distribution, or prescription filling of controlled substances prescribed via fraudulent picture IDs because it does not recognize the identities of the individuals on these IDs as fakes. As an example, some controlled substances are distributed in the USA to recovering drug users via the state sponsored programs. However, as described further below, non-drug users presenting different fake IDs to different medical providers can obtain such controlled substances for the purposes of reselling the drugs on the black market.

To control distribution, governmental agencies mandate that certain safeguards be maintained in the industry. For instance, under the current proposal, all prescriptions will be written online by 2021 via a secured web base interface and a centralized server. However, despite this safeguard, fraudulent obtainment of personal identification data is highly probable, and there is a great chance that a prescription for controlled substances may be filled without proper monitoring and controlled substances be resold on the black market.

Some controlled substances (e.g., Suboxone) are supplied to people undergone through detoxification from drug addiction to prevent relapse via a very popular government funded program free of charge. Relapse rate after detoxification from the opioid abuse is estimated at 94%. These controlled substance prevents a relapse after the detoxification. Unfortunately, such relapses often result in overdose deaths because, at the time of relapse, relapsing addicts often take their last known to them dose without realizing that their tolerance to opioids became much lower during the detoxification. Typical cause of death due to opioid overdose is respiratory paralysis. For these individuals, the controlled medication which prevents the relapse is prescribed for life.

Unfortunately, depending on the quantity taken, the same medications can cause euphoric effects and can cause addiction on their own with a larger dose. Moreover, if the same medication is mixed with heroin, it potentiates the heroin effect.

Given today's prevalence of identity fraud and absence of reliable systems for people's identification, these medications may still be obtained by people who are not drug addicts but who know how to fake the urine test for opioids. Once the person tests positive for opioids, all that is needed for him/her to receive the controlled substance is to make a request to be placed into the program. As of this writing, once the fraudulent non-drug user is placed into the program, he/she will receive 30 tablets a month, one tablet for each day, every month for life to be available to sell on the black market. More tablets can be obtained if the fraudulent non-drug user uses several fake identities and fake IDs, registers using these IDs for the program from different medical providers, and then receives the drugs from different pharmacies. There is no way at present to identify these individuals and/or to detect them at the time when they obtain medications from pharmacies.

Nowadays, it is easier than ever to obtain fake IDs with the same picture, but with different first and last names and dates of birth, especially if information is mined and hacked. Because present biometric technologies are slow and are not 100% accurate, they are expensive to maintain and, therefore, they are underutilized. Further, a fake patient with a fake patient ID can get a prescription for an opioid in NY, fill it in NJ, and Department of Health in NY will not even know who obtained the medication. A doctor's office or a pharmacy has no tools to authenticate the information from the IDs presented at the time when the patient presents at their location.

This is especially true if the pharmacy receives a valid doctor's prescription. All they have to do is validate the method of payment and make a copy of the presented picture ID. The I-STOP/PMP system at the time of prescription fill will recognize these fake individuals receiving their single monthly supply as legitimate individuals unrelated to each other and will not be able to detect the obtainment of the medications via fraudulent IDs. If there is no insurance information the pharmacy will get paid in cash, and the fraudulent individual will be free to re-sell the drugs on the black-market times more expensive.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates the content and structure of Unique Patient Identification Entity.

FIG. 2 illustrates a system and method of integrated patient, or person unique identity management, in accordance with one embodiment of the present invention.

Below is a clear example of the invention use in pharmaceutical industry.

### DETAILED DESCRIPTION OF ILLUSTRATIVE EMBODIMENTS

Various aspects of the illustrative embodiments may be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted, or simplified in order not to obscure the illustrative embodiments.

Various operations will be described as multiple discrete operations, in turn, in a manner that is most helpful in understanding the present invention; however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise. The word “patient” is applicable in relation to the healthcare industry, whereas word “personal” is applicable in other industries alike, instead of the word “patient”.

The patient's unique identity entity consists of two independent identification data objects such as patient's ID tag (PIT) formed from person individual data and a biometric data. Embodiments of the invention are discussed below with reference to the Figures. However, those skilled in the art will readily appreciate that the detailed description given herein with respect to these figures is for explanatory purposes as the invention extends beyond these limited embodiments.

For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. In addition, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

It is well settled law, as early as 1939, that words of approximation are not indefinite in the claims even when such limits are not defined or specified in the specification.

Hence, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

As used herein, the phrase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phrase “consisting essentially of” limits the scope of a claim to the specified elements or method steps, plus those that do not materially affect the basis and novel characteristic(s) of the claimed subject matter. Moreover, for any claim of the present invention which claims an embodiment “consisting essentially of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of” and “consisting essentially of” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary, varieties of optional components are described to illustrate the wide variety of possible embodiments of the present invention.

As is well known to those skilled in the art many careful considerations and compromises typically must be made when designing for the optimal manufacture of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A “computer” may refer to one or more apparatus and/or one or more systems that are capable of accepting a structured input, processing the structured input according to prescribed rules, and producing results of the processing as output. Examples of a computer may include: a computer; a stationary and/or portable computer; a computer having a single processor, multiple processors, or multi-core processors, which may operate in parallel and/or not in parallel; a general purpose computer; a supercomputer; a mainframe; a super mini-computer; a mini-computer; a workstation; a micro-computer; a server; a client; an interactive television; a web appliance; a telecommunications device with internet access; a hybrid combination of a computer and an interactive television; a portable computer; a tablet personal computer (PC); a personal digital assistant (PDA); a portable telephone; application-specific hardware to emulate a computer and/or software, such as, for example, a digital signal processor (DSP), a field-programmable gate array (FPGA), an application specific integrated circuit (ASIC), an application specific instruction-set processor (ASIP), a chip, chips, a system on a chip, or a chip set; a data acquisition device; an optical computer; a quantum computer; a biological computer; and generally, an apparatus that may accept data, process data according to one or more stored software programs, generate results, and typically include input, output, storage, arithmetic, logic, and control units.

Those of skill in the art will appreciate that where appropriate, some embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Where appropriate, embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

“Software” may refer to prescribed rules to operate a computer. Examples of software may include: code segments in one or more computer-readable languages; graphical and or/textual instructions; applets; pre-compiled code; interpreted code; compiled code; and computer programs.

The example embodiments described herein can be implemented in an operating environment comprising computer-executable instructions (e.g., software) installed on a computer, in hardware, or in a combination of software and hardware. The computer-executable instructions can be written in a computer programming language or can be embodied in firmware logic. If written in a programming language conforming to a recognized standard, such instructions can be executed on a variety of hardware platforms and for interfaces to a variety of operating systems. Although not limited thereto, computer software program code for carrying out operations for aspects of the present invention can be written in any combination of one or more suitable programming languages, including an object oriented programming languages and/or conventional procedural programming languages, and/or programming languages such as, for example, Hyper text Markup Language (HTML), Dynamic HTML, Extensible Markup Language (XML), Extensible Stylesheet Language (XSL), Document Style Semantics and Specification Language (DSSSL), Cascading Style Sheets (CSS), Synchronized Multimedia Integration Language (SMIL), Wireless Markup Language (WML), Java™, Jini™, C, C++, Smalltalk, Perl, UNIX Shell, Visual Basic or Visual Basic Script, Virtual Reality Markup Language (VRML), ColdFusion™ or other compilers, assemblers, interpreters or other computer languages or platforms.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

A network is a collection of links and nodes (e.g., multiple computers and/or other devices connected together) arranged so that information may be passed from one part of the network to another over multiple links and through various nodes. Examples of networks include the Internet, the public switched telephone network, the global Telex network, computer networks (e.g., an intranet, an extranet, a local-area network, or a wide-area network), wired networks, and wireless networks.

The Internet is a worldwide network of computers and computer networks arranged to allow the easy and robust exchange of information between computer users. Hundreds of millions of people around the world have access to computers connected to the Internet via Internet Service Providers (ISPs). Content providers (e.g., website owners or operators) place multimedia information (e.g., text, graphics, audio, video, animation, and other forms of data) at specific locations on the Internet referred to as webpages. Websites comprise a collection of connected, or otherwise related, webpages. The combination of all the websites and their corresponding webpages on the Internet is generally known as the World Wide Web (WWW) or simply the Web.

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

It will be readily apparent that the various methods and algorithms described herein may be implemented by, e.g., appropriately programmed general purpose computers and computing devices. Typically, a processor (e.g., a microprocessor) will receive instructions from a memory or like device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself

The term “computer-readable medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, removable media, flash memory, a “memory stick”, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, 3G.

Where databases are described, it will be understood by one of ordinary skill in the art that (i) alternative database structures to those described may be readily employed, (ii) other memory structures besides databases may be readily employed. Any schematic illustrations and accompanying descriptions of any sample databases presented herein are exemplary arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, an object-based model could be used to store and manipulate the data types of the present invention and likewise, object methods or behaviors can be used to implement the processes of the present invention.

A “computer system” may refer to a system having one or more computers, where each computer may include a computer-readable medium embodying software to operate the computer or one or more of its components. Examples of a computer system may include: a distributed computer system for processing information via computer systems linked by a network; two or more computer systems connected together via a network for transmitting and/or receiving information between the computer systems; a computer system including two or more processors within a single computer; and one or more apparatuses and/or one or more systems that may accept data, may process data in accordance with one or more stored software programs, may generate results, and typically may include input, output, storage, arithmetic, logic, and control units.

A “network” may refer to a number of computers and associated devices that may be connected by communication facilities. A network may involve permanent connections such as cables or temporary connections such as those made through telephone or other communication links. A network may further include hard-wired connections (e.g., coaxial cable, twisted pair, optical fiber, waveguides, etc.) and/or wireless connections (e.g., radio frequency waveforms, free-space optical waveforms, acoustic waveforms, etc.). Examples of a network may include: an internet, such as the Internet; an intranet; a local area network (LAN); a wide area network (WAN); and a combination of networks, such as an internet and an intranet.

As used herein, the “client-side” application should be broadly construed to refer to an application, a page associated with that application, or some other resource or function invoked by a client-side request to the application. A “browser” as used herein is not intended to refer to any specific browser (e.g., Internet Explorer, Safari, Firefox, or the like), but should be broadly construed to refer to any client-side rendering engine that can access and display Internet-accessible resources. A “rich” client typically refers to a non-HTTP based client-side application, such as an SSH or CFIS client. Further, while typically the client-server interactions occur using HTTP, this is not a limitation either. The client server interaction may be formatted to conform to the Simple Object Access Protocol (SOAP) and travel over HTTP (over the public Internet), FTP, or any other reliable transport mechanism (such as IBM® MQSeries® technologies and CORBA, for transport over an enterprise intranet) may be used. Any application or functionality described herein may be implemented as native code, by providing hooks into another application, by facilitating use of the mechanism as a plug-in, by linking to the mechanism, and the like.

Exemplary networks may operate with any of a number of protocols, such as Internet protocol (IP), asynchronous transfer mode (ATM), and/or synchronous optical network (SONET), user datagram protocol (UDP), IEEE 802.x, etc.

Embodiments of the present invention may include apparatuses for performing the operations disclosed herein. An apparatus may be specially constructed for the desired purposes, or it may comprise a general-purpose device selectively activated or reconfigured by a program stored in the device.

Embodiments of the invention may also be implemented in one or a combination of hardware, firmware, and software. They may be implemented as instructions stored on a machine-readable medium, which may be read and executed by a computing platform to perform the operations described herein.

More specifically, as will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

In the following description and claims, the terms “computer program medium” and “computer readable medium” may be used to generally refer to media such as, but not limited to, removable storage drives, a hard disk installed in hard disk drive, and the like. These computer program products may provide software to a computer system. Embodiments of the invention may be directed to such computer program products.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated.

It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Unless specifically stated otherwise, and as may be apparent from the following description and claims, it should be appreciated that throughout the specification descriptions utilizing terms such as “processing,” “computing,” “calculating,” “determining,” or the like, refer to the action and/or processes of a computer or computing system, or similar electronic computing device, that manipulate and/or transform data represented as physical, such as electronic, quantities within the computing system's registers and/or memories into other data similarly represented as physical quantities within the computing system's memories, registers or other such information storage, transmission or display devices.

Additionally, the phrase “configured to” or “operable for” can include generic structure (e.g., generic circuitry) that is manipulated by software and/or firmware (e.g., an FPGA or a general-purpose processor executing software) to operate in a manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks.

In a similar manner, the term “processor” may refer to any device or portion of a device that processes electronic data from registers and/or memory to transform that electronic data into other electronic data that may be stored in registers and/or memory. A “computing platform” may comprise one or more processors.

Embodiments within the scope of the present disclosure may also include tangible and/or non-transitory computer-readable storage media for carrying or having computer-executable instructions or data structures stored thereon. Such non-transitory computer-readable storage media can be any available media that can be accessed by a general purpose or special purpose computer, including the functional design of any special purpose processor as discussed above. By way of example, and not limitation, such non-transitory computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

While a non-transitory computer readable medium includes, but is not limited to, a hard drive, compact disc, flash memory, volatile memory, random access memory, magnetic memory, optical memory, semiconductor based memory, phase change memory, optical memory, periodically refreshed memory, and the like; the non-transitory computer readable medium, however, does not include a pure transitory signal per se; i.e., where the medium itself is transitory.

In line with the current specification, a PIT is formed. The PIT is a specific integer number formed by converting the person's full name, birthday and eye color by special following algorithm: the person's full name is the set of names by which an individual is known and that can be introduced as a word-group, with the understanding that, taken together, they all relate to that one individual. The full name is preferably presented in the form of: first/given, middle, and last/family/surname.

The following is an example of PIT formation: From the full name string all non-alphanumeric characters are removed; The full name string value is converted to uppercase; The first character's coefficient number is calculated as the sum of the ASCII values of the first letters of each word in the full name string; From the full name string all spaces are removed; The first part of the PIT number then consists of the sum of ASCII value of each character in the full name string.

The second part of patient's PIT number preferably consists of a string joined of values from the day number plus the month number and the year of the birthday.

The third part of the patient's PIT number is formed from the length value of the full name string plus the first character's coefficient number.

The fourth part of the PIT number is based on a biometric data coefficient that is predetermined based on the bio characteristic of the person. For example, if the biometric data is eye color, the eye color code will be based on eye color chart, preferably as follows:

### BLK Black=1

### BLU Blue=2

### BRO Brown=3

### GRY Gray=4

### GRN Green=5

### HAZ Hazel=6

### MAR Maroon=7

### PNK Pink=8

### DIC Dichromatic=9

### SPC Spectrum=10

Thus, for example, for the person with full name “James Bob Smith”, with a date of birth May 15 1926 and gray eye color, the PIT will be: 9681926515132234, where:

968—is the first part of the patient's PIT number and consists of the sum of ASCII value of each character in the full name string;
1926515—is the second part of PIT number and consists of a string of values from the day number plus the month number and the year of the birthday;
13223—is the third part of the PIT number and is formed from the length value (=13) of the full name string and joined first character's coefficient number (74(J)+66(B)+83(S)=223); and
4—is the fourth part of the patient's PIT and corresponds to the eye color.

Among other biometric data, ear images may be used which may be acquired in a similar manner to face images, and a number of researchers have suggested that the human ear is unique enough to each individual to allow practical use as a biometric. For obtaining biometric data of a person's ear can be used a simple video or digital camera. Ear biometric system consists of ear detection and ear recognition modules.

There are several algorithms for image digitizing of human ear. Most popular shape-finding algorithm called “image ray transform,” which boasts 99.6 percent accuracy, according to a study presented at the IEEE Fourth International Conference on Biometrics Sep. 29, 2014. The outer ear may prove to be one of the most accurate and least intrusive ways to identify people.

We propose that the analysis of the curve of the ear's helix is the most reliable anatomical structure (antihelix, tragus, antitragus, inter-tragic incisura, and the ear lobule) when both left and right ears were paired together. In this approach rate of identification should be close to 100%.

Since the unique identification, the UPIE, consists of two independent identification data objects the requirements to high accuracy can be reduced. This approach boosts performance and reduces costs of measuring.

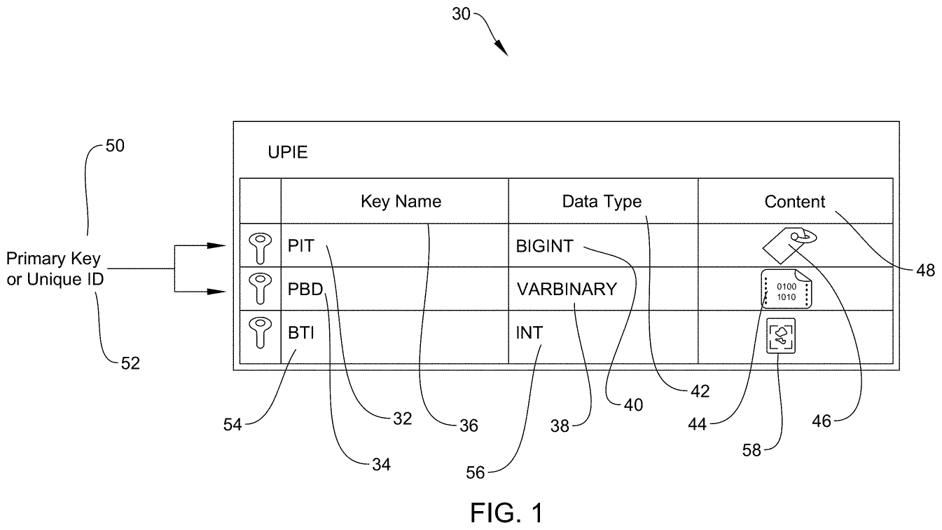

Referring now to FIG. 1, as the example outlined for healthcare use, illustrated is The Unique Patient Identification Entity (UPIE) 30 having a PIT 32, as well as patient's biometric data (PBD) 34, where 44 is biometric data that is used to uniquely identify each person. However, uniqueness is only guaranteed when the PIT 32 and PBD 34 are combined. This combination can be used in the nationwide Big Table as a Primary Key (PK) 50 or unique ID 52 which consists from two columns PIT 32 and PBD 34. Each of their column is Candidate Key that can uniquely identify a patient's record in a Big Table. The PIT 32 column can be in integer-based data type 42 with indexing that improves the speed of data retrieval operations to make select query run faster. Shown are data type 42, bigint 40, and varbinary 38. Key Name 36 is shown. Content 48 has PIT 32, PBD 44, and integer data Content 58. BTI 54 column is used to identify biometric input devices and their algorithms which can be standardized and unified for national use. Int 56 is Integer Content.

Because each single column's data can be duplicated but the combination values of these columns cannot be duplicated, the searching by integer-based data in the first column of PIT 32 will reduce the amount of records for searching binary-based data in second column of PBD 34 data. The combination of PIT 32 and PBD 34 values called Unique Patient Identification Entity (UPIE) 30 and can be used in the various types of relational and non-relational databases.

The Unique Patient Identification Entity (UPIE) 30 is comprises of both, PIT 32 and PBD 34 together, and forms a pair as unique identity entity of person that uniquely identifies him among all other personal entities. Because value objects are immutable, it automatically fulfills the first requirement of identity: immutability. Because both values of objects, as a pair, are unique, they give it the second component of identity: uniqueness. Thereby, UPIE 30 is immutable and globally unique.

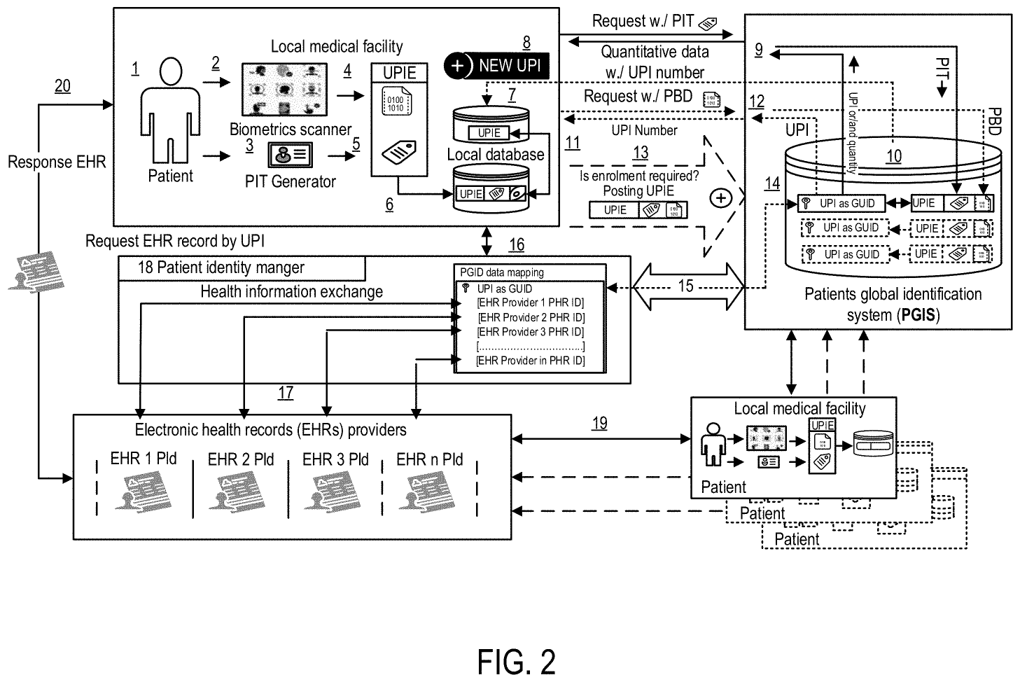

Referring now to FIG. 2, the figure illustrates Patients Global Unique Identification System (PGUIS) of the present invention. The uniqueness of this system architecture can be seen as simple cost-effective way to manage patients' identities across data sources, and the Unique Patient Identification Entity (UPIE) can be formed independently of the global system in any remote location, for example, in a medical facility. The images of a person's (patient's) ears (1) are captured with a high-resolution camera and is stored in the files. The ear region of each image is located and extracted by the recognition system (2). On the next phase, the texture of the ear image is extracted (4) using linear time-invariant (LTI) filter responses and converts it to virtual extraction feature (acquired bio-metric data) for right and left ears, respectively. Extraction features of both right and left ears paired together to the patient binary biometric data file (PBD).

Meanwhile, the program that calculates a PIT processes the patient's application data. When PIT and PBD objects are created they are combined in the unique person identification entity (UPIE). The UPIE is stored in the local database (7).

Patient identity management (PIM). Electronic health care data are constantly being generated and linked across multiple systems, including electronic health records (EHRs), patient registries, and claims databases. In general, every system assigns its own identifier to each patient whose data it maintains. This makes it difficult to track patients across multiple systems and identify duplicate patients when different systems are linked. Efforts to address these challenges are complicated by the need to protect personal privacy and security.

Several standard development organizations are involved in the development of PIM strategies and standards. Major organizations currently include: Integrating the Healthcare Enterprise; Health Level Seven International; and The Regentrify Institute, Inc.

In accordance with the Patient Identity Management (PIM) system of the present invention, PGUIS can be used as the foundational platform for a centralized countrywide level electronic database of all patients known as Central Data Repository (CDR) (containing all relevant data including biometrics in uniquely designed formats, all-time updated) for global level identity management.

To obtain a Unique Patient Identification (UPI) number from the global database, a special lightweight search algorithm is used as below.

The local medical facility application sends PIT (8), see FIG. 2, to the global patient system (PGIS) as an integer number (9). Because the PIT number itself can be unique the PGIS response time to a request will be short. The PGIS fast response consists of small value of data (such as XML, JSON, or plain text) of two numbers: 1) Total number of records in the PGIS Big Table for requested PIT number, and 2) the GUID (Globally Unique ID) number of PIT record in the PGIS Big Table with the following variables: 1) if total number of records for requested PIT number equal 1, or 2) zero (null), or 3) if total number of records is more than 1. Depending on the value of the total number of records received from the PGIS Big Table for requested PIT number, the following processes are performed:

If total number of records in the PGIS Big Table for requested PIT number equals to zero, the person enrolment process is performed: local medical facility sends UPIE entity (13) to the PGIS and retrieve response by UPI number (10). If total number of records in the PGIS Big Table for requested PIT number equals to 1, the local medical facility receives response by UPI number (8). If total number of records in the PGIS Big Table for requested PIT number more than 1, the local medical facility sends request with PBD (12) and receives the response by UPI number (11).

When the number of UPI have been obtained, local facility application updates UPIE record in local database with (7) UPI and utilizes UPI to make a request to EHR companies and providers (16) of electronic health records (EHR) by UPI.

On this proposed approach, the patient identity management system (PIMS) is a part of the PDGUIS. The PIMS database contains records in which the unique patient identifier corresponds to the local identification number of patient health record on the provider's site.

The electronic health care providers (17), see FIG. 2, make additional check points for EHR using direct connection to the health information exchange system HIES (15). The health information exchange system (HIES) intended to share electronic patient health (ePHI) records across disparate health care applications. This platform provides inter-mobility to exchange patient data produced by health care applications (19) with other applications that consume and use the data, such as EHRs, healthcare global ID verification for medical equipment, electronic record management and management of patient identity across data sources. The national patient identification system would identify patients, link medical records, and allow broad sharing, monitoring, research and analysis using computerized medical records linked through the nationwide health information network (NHIN).

### Patients Global Unique Identification System (PGUIS)

The uniqueness of this system's architecture is allowing for a simple, cost-effective way to manage patient identity across data sources. In addition, the Unique Patient Identification Entity (UPIE) containing PIT and PBD data objects is formed independently of the global system in any remote location (i.e. medical facility).

The biometric information from an individual (1) is captured (2) and stored in the file/s as a binary patient biometric data file (PBD).

In another aspect of the present invention, a method for preventing pharmaceutical fraud is provided using the above-described integrated identity management system. Specifically, the following steps are performed to prevent pharmaceutical fraud:

Patient (1) comes to see a doctor in a local medical facility and verification process is initiated;

As shown in FIG. 2, the local medical facility sends out PIT to PGIS and receives one of the following:
a) GUID Number and PIT Records equal to 0. If total number of records in the PGIS Big Table for requested PIT number equals to zero, the person enrolment process is performed: local medical facility sends UPIE entity (13) to the PGIS and receives response by UPI number (10).
b) GUID Number and PIT Records equal to 1. If total number of records in the PGIS Big Table for requested PIT number equals to 1, the local medical facility receives response by UPI number (8).
c) GUID Number and PIT Records equals to more than 1. If total number of records in the PGIS Big Table for requested PIT number is more than 1, the local medical facility sends request with PBD (12) and receives the response by UPI number (11).

When the number of UPI have been obtained, the local facility application updates UPIE record in local database with (7) UPI and utilizes UPI to make a request to EHR companies and providers (16) of electronic health records (EHR) by UPI.

The medical facility then sends the prescription and, associated with it, UPIE first to a controlling governmental body and waits for the response thereby creating the checkpoint at which the controlling body can very quickly electronically notify the medical facility of any identity conflict. If there is no identity conflict, the medical facility receives an automatic message to proceed by sending the electronic prescription to the pharmacy with the copy of the message that there are no identity conflicts.

Before the patient or his/her representative arrives at the pharmacy to pick up the prescription, the pharmacist receives the verification (controlling body's message) and the data to make sure that the controlled substance and its UPIE have no identity conflict. Once this is verified, the prescription can be filled.

When the above steps of the inventive method are followed, no controlled substance is filled before the receipt of a copy of the message that there is no identity conflict with the prescription medication and the associated UPIE. An example of the identity conflict may be when the same prescription is written for the same UPIE within an unusually short amount of time. If the medical facility receives the message that there is an identity conflict, the prescription is not generated, and, of course, not filled.

In the preceding specification, the invention has been described with reference to specific exemplary embodiments thereof. It will however, be evident that various modifications and changes may be made thereto without departing from the broader spirit and scope of the invention as set forth in the claims that follow.

We claim as follows:

1. A computer-implemented method of preventing pharmaceutical fraud, the method comprising the steps of:
using a first computer component associated with a local facility to process a set of personal data of a person requesting a prescription to generate a first personal identification tag;
sending said generated first personal identification tag to a database comprising a plurality of personal identification tags;
using a second computer component associated with said database to compare said first personal identification tag to said plurality of personal identification tags within said database, and sending a first identifying response to said local facility;
using a third computer component also associated with said local facility to request electronic health records based on said first identifying response;
using a fourth computer component also associated with a local facility to request an approval from a controlling entity for a prescription based on said first identifying response and said electronic health records;
using a fifth computer component associated with said controlling entity to evaluate said first identifying response, said request for approval and said electronic health records, and to generate one of an approval response and a rejection response, wherein an approval response is generated when no conflicts are found with respect to the first identifying response, and wherein a rejection response is generated when at least one conflict is found with respect to the first identifying response; and
using a sixth computer component associated with said local facility to generate said prescription.

2. The computer-implemented method according to claim 1, wherein said first personal identification tag comprises a specific integer number produced using a seventh computer component associated with said local facility from said set of personal data by first converting a first type of personal data, wherein the first type of personal data comprises alphanumeric and non-alphanumeric characters, into a converted personal data string by removing all non-alphanumeric characters; next converting the converted personal data string to a second personal data string wherein all alphanumeric characters are in uppercase, then converting the second personal data string to a number string with at least four parts, wherein a first part is a number value which is calculated as the sum of ASCII values of first letters of each word in the converted personal data string, wherein a second part of said number string comprises values representing a selected day, wherein a third part of said number string is calculated from a length value of the words in the converted personal data string plus a coefficient number, wherein a fourth part of said number string is based on a physical characteristic of said person, and wherein said number string is used to identify said person.

3. The computer-implemented method of claim 2 wherein a portion of the first type of personal data is the full name of a person.

4. The computer-implemented method of claim 3 wherein alphanumeric characters in the full name are calculated based on cultural alternative characters table for other countries.

5. The computer-implemented method of claim 4 wherein the selected day is a birthday in a form of a combination of a day number, a month number, and a year of the birthday.

6. The computer-implemented method of claim 5 wherein a portion of the first type of personal data is a number representing an eye color of said person.

7. The computer-implemented method of claim 6 wherein the eye color of said person is represented by a code based on an eye color chart.

8. The computer-implemented method according to claim 1, further comprising a step of using an eighth computer component to process a set of biometric data to form a person's binary biometric data by first obtaining a person's biometric data and then converting said biometric data into said person's binary biometric data and to combine said first personal identification tag with said person's binary biometric data to generate a unique person's identifier.

9. The computer implemented method according to claim 8 wherein said step of using the eighth computer component to process said set of biometric data comprises analyzing a curve of said person's ear helix and converting the results of said analysis into a second number string.

10. The computer-implemented method according to claim 1, wherein said first identifying response is one of 0, 1 and more than 1.

11. The computer-implemented method according to claim 10, wherein when said first identifying response is 0, said method further comprises a step of saving said person's first personal identification tag in said database.

12. The computer-implemented method according to claim 10, wherein when said first identifying response is 1, said method further comprises a step of retrieving identifying information for said person from said database.

13. The computer-implemented method according to claim 10, wherein when said first identifying response is more than 1, said method further comprises a step of adding a biometric information of said person to said first personal identification tag to retrieve identifying information of said person from said database.
